(19) United States
(12) Patent Application Publication
(10) Pub. No.: US 2004/0148028 A1
(43) Pub. Date: Jul. 29, 2004

(54) Artificial disc replacement (ADR) extraction methods and apparatus

(76) Inventors: Bret A. Ferree, Cincinnati, OH (US); Alex B. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/741,290

(22) Filed: Dec. 19, 2003

Related U.S. Application Data
(60) Provisional application No. 60/434,894, filed on Dec. 19, 2002.
(60) Provisional application No. 60/478,321, filed on Jun. 13, 2003.

(51) Int. Cl. A61F 2/44

(52) U.S. Cl. 623/17.11; 623/17.16; 606/61

(57) ABSTRACT

Methods and apparatus facilitate artificial disc replacement (ADR) removal. One aspect of the invention fastens ADR removal instruments to the ADR. The robust connection between the novel removal tools and the ADR allow the application of large forces. A second aspect of the invention provides special osteotomes, chisels, saws, and drills to release the ADR from the vertebrae. A guide system minimizes destruction of the vertebrae. A third aspect of the invention is directed to marking the location of spikes, keels, or other ADR projections on the front or other visible portion of the ADR. A fourth aspect of the invention involves the use of optional releasable spikes, fins, keels, or other projections. A fifth aspect of the invention resides in cement removal techniques and tools to remove cemented ADRs.

### REFERENCE TO RELATED APPLICATIONS

This application claims priority from U.S. Provisional Patent Application Serial No. 60/434,894, filed Dec. 19, 2002 and 60/478,321, filed Jun. 13, 2003, the entire content of each being incorporated herein by reference.

### FIELD OF THE INVENTION

This invention relates generally to artificial intervertebral disc replacements (ADRs) and, more particularly, to apparatus and methods for extracting ADRs.

### BACKGROUND OF THE INVENTION

Premature or accelerated intervertebral disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Implanted artificial disc replacements (ADRs) will, at times, however, require removal and revision. For example, implanted ADRs may need to be replaced if the ADR wears out or the ADR becomes infected.

Prior-art ADRs have not considered a system to facilitate ADR extraction. Extraction of prior-art ADRs with current methods and instruments risks destruction of the vertebrae above and below the disc replacement. Damage to the vertebrae adjacent to the ADR may lead to spinal cord injury or excessive bleeding. Furthermore, the damage to the vertebrae could prohibit the reinsertion of a new ADR.

### SUMMARY OF THE INVENTION

This invention is directed to methods and apparatus for facilitating artificial disc replacement (ADR) removal. The system is intended to make ADR revision safer while increasing the chances of reinserting a second ADR. The system helps preserve the vertebrae above and below the ADR.

The preferred embodiments include various aspects. One aspect of the invention fastens ADR removal instruments to the ADR. Unlike the weak connection between current ADR insertion instruments and the ADR, the robust connection between the novel removal tools and the ADR allow the application of large forces. Furthermore, insertion tools are designed for applying forces toward the spine rather than away from the spine. Large forces may be necessary to pull the ADR from the vertebrae. The robust connection also minimizes the risk to adjacent soft tissues, such as the great vessels, by preventing the extraction tool from prematurely disconnecting from the ADR.

Any rigid coupling mechanism between extraction tool and the ADR that allows the application of force away from the spine can be used according to the invention. For example, the extraction tool could be threaded into the ADR. Alternatively, a coupling mechanism between a lipped slot and a lipped projection could be used. In either embodiment, a slotted slap hammer could be used over the shaft of the instruments. The instrument connected to the ADR could also be connected to a threaded puller like instrument that cooperates with the vertebrae to apply force that pulls the ADR away from the spine.

A second aspect of the invention provides special osteotomes, chisels, saws, and drills to release the ADR from the vertebrae. A guide system minimizes destruction of the vertebrae. The guide controls the course of the instruments to remove only a small portion of the vertebrae directly adjacent to the ADR. The use of prior-art chisels removes excessive bone as the chisel wonders from the hard ADR and into the soft bone of the vertebrae. Attempts to pry ADRs from the vertebrae with current tools damage a significant portion of the vertebrae. For example, twisting metal instruments to “cam” the ADR off the vertebrae will damage the soft bone of the vertebrae. The instruments that are used to cut a path between the vertebrae and the ADR can be guided by the rigidly attached extractor tool described in the paragraph above. Novel cutting tools for use without the guide, have depth stops to prevent inadvertent penetration into the spinal canal.

A third aspect of the invention is directed to marking the location of spikes, keels, or other ADR projections on the front or other visible portion of the ADR. When properly placed in the bone of the vertebrae, the projections from the ADR are not visible. The visible, anterior surface of the ADR will be marked with icons that show the location, size, and type of projection. Locating the type and location of the projection helps the surgeon select the type of cutting tool to release the ADR from the vertebrae. Knowing the location of the projections also helps the surgeon select the method to extract the particular ADR. Fluoroscopy or other navigational device could also help the surgeon identify projections from the ADR. Fluoroscopy or other navigational device could direct the surgeon in removing bone from around the projections.

A fourth aspect of the invention involves the use of optional releasable spikes, fins, keels, or other projections. For example, the keels or other projections could be notched or otherwise pre-stressed to facilitate release of the projections from the ADR. The projections could remain embedded in the vertebra. A second ADR with a different pattern of projections could be inserted around the projections that remain in the vertebrae. Alternatively, the projections could be removed from the vertebrae after the majority of the ADR was removed. Modular keels or other projections could be used. For example, modular keels could be removed from the ADR before the ADR is removed from the vertebrae.

A fifth aspect of the invention resides in cement removal techniques and tools to remove cemented ADRs. For example cement chisels could be guided by the extraction tool. Cement removal tools used without the guide have depth stops to prevent projection into the spinal canal. Ultrasonic instruments or other devices used to remove polymethylmethacrylate (PMMA) during revision hip or knee replacement surgeries, could also be used or adapted for use in revision ADR surgery.

### BRIEF DESCRIPTION OF THE DRAWINGS

FIG. 1A is a sagittal cross-section of an ADR with an extraction tool threaded to the front of the ADR;

FIG. 1B is a sagittal cross-section of an ADR and an extraction tool with an alternative attachment mechanism;

FIG. 1C is a sagittal cross-section of an ADR and an extraction tool with an alternative locking mechanism;

FIG. 1D is a sagittal cross-section of the spine, an ADR, and a novel ADR puller tool;

FIGS. 1E-1H show the way in which single tools or multiple tools may be inserted between implanted endplates for ADR removal;

FIG. 2A is a sagittal cross section of an ADR and a guided chisel;

FIG. 2B shows view of the top of an ADR with a keel and a novel instrument designed to fit around the keel;

FIG. 2C shows is a view of the top of an ADR with two rows of spikes and an embodiment of the tool drawn in FIG. 2B designed to cut around the spikes;

FIG. 2D is a sagittal cross section of an ADR, a guide, and a tool designed to cut around the top of the ADR and the sides of a keel;

FIG. 2E is a coronal cross-section of an ADR and the cutting tool draw in FIG. 2D;

FIG. 2F is a sagittal cross-section of an ADR and an alternative cutting tool and cutting guide;

FIG. 2G is view of the front of the superior endplate of an ADR and the embodiment of the cutting guide drawn in FIG. 2F;

FIG. 3A is the view of the front of an ADR with novel marks that locate the size, type, and position of projections from the ADR;

FIG. 3B is the view of the front of an ADR with markings to indicate the size and location of ADR spikes;

FIG. 4A is a lateral view of an ADR with novel stress risers to facilitate separation of projections from the ADR;

FIG. 4B is a coronal cross section of an ADR with novel modular keels;

FIG. 4C is a view of the front of the ADR drawn in FIG. 4B;

FIG. 5A is a view of the top of the bottom ADR endplate or the view of the bottom of the top endplate;

FIG. 5B is a view of the side of the embodiment of the ADR drawn in FIG. 5A; and

FIG. 5C is an exploded view of the embodiment of the ADR drawn in FIGS. 5A and 5B.

### DETAILED DESCRIPTION OF THE INVENTION

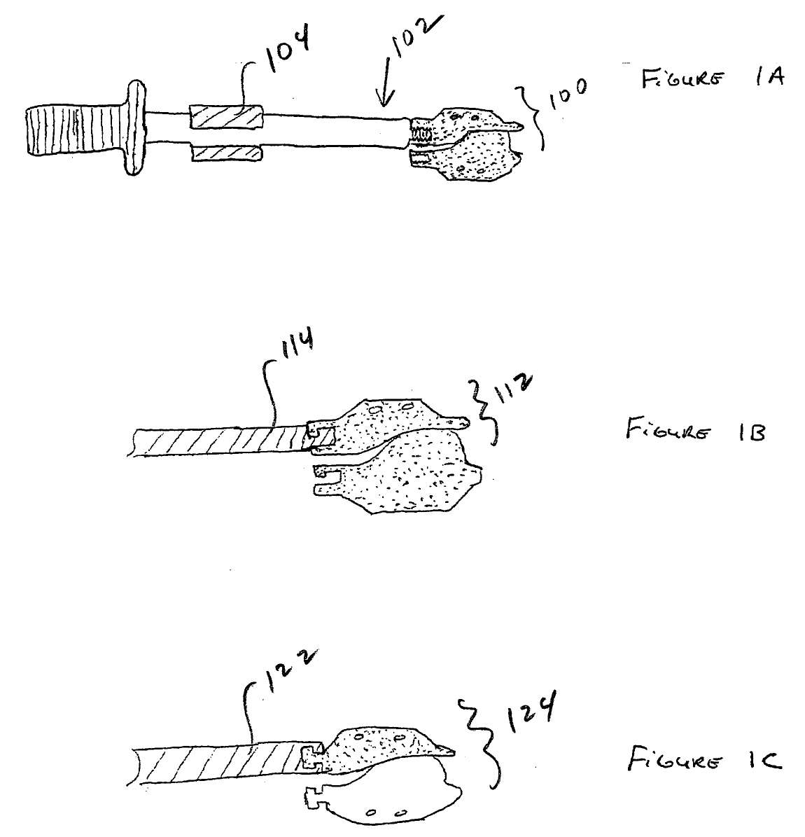

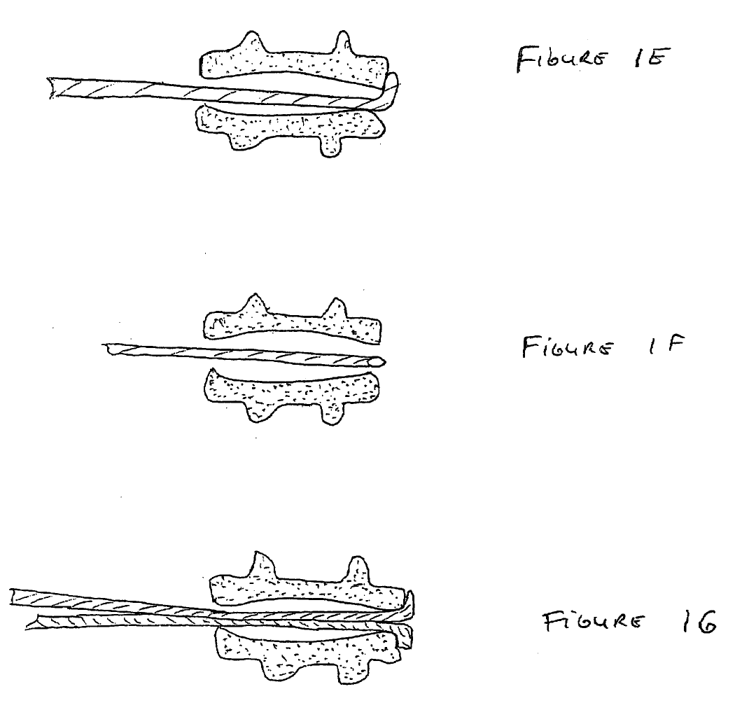

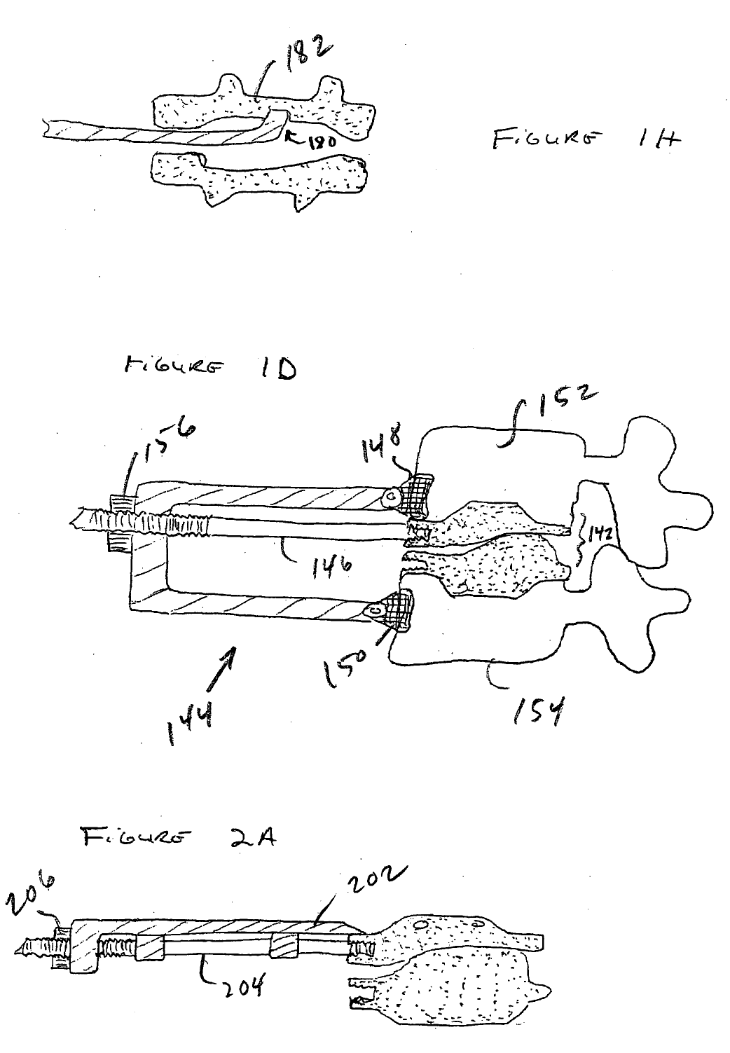

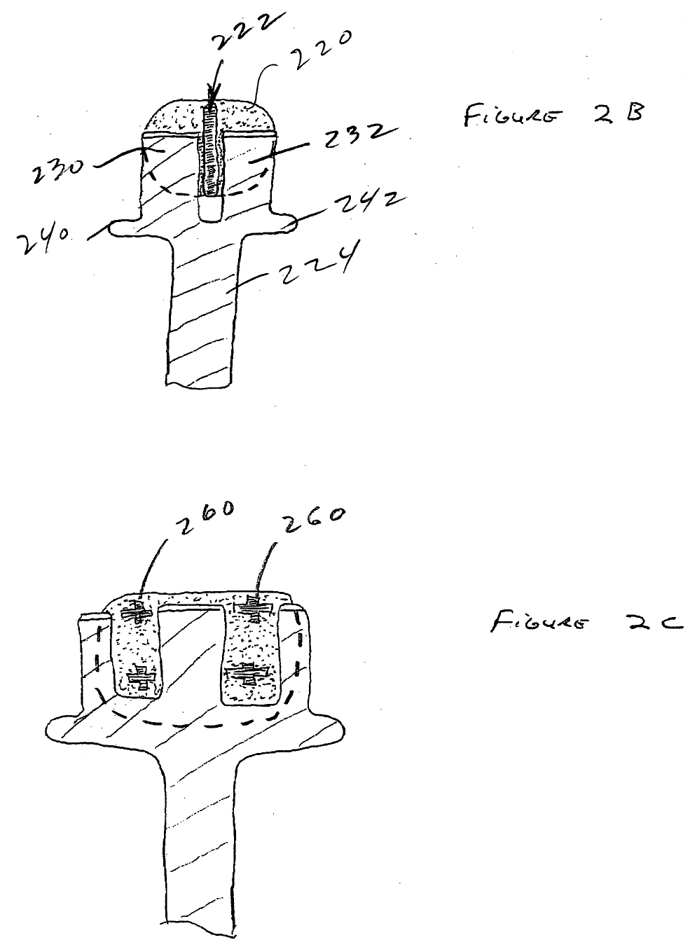

Now turning to the drawings, FIG. 1A is a sagittal cross section of an ADR 100 with an extraction tool 102 threaded to the front of the ADR. A slap hammer is shown at 104. FIG. 1B is a sagittal cross section of an ADR 112 and an extraction tool 114 with an alternative attachment mechanism. A projection from the shaft of the extraction tool 114 is placed into a slot in the ADR. The extraction tool is rotated within the slot of the ADR. The projection from the extractor tool rotates behind a portion of the ADR. In the preferred embodiment, the slot is rotated 90 degrees to that shown in the drawing. The slot was drawn horizontally to better illustrate the coupling mechanism.

FIG. 1C is a sagittal cross section of an ADR and an extraction tool with an alternative locking mechanism. A slot in the extraction tool 122 couples with a projection from the ADR 124. FIG. 1D is a sagittal cross section of the spine, an ADR 142, and a novel ADR puller tool 144. The shaft 146 of the tool that is threaded into the ADR fits through a hole in the puller tool. The puller tool has feet 148, 150 that fit over anterior aspect of the vertebrae 152, 154 or the other ADR endplate. Rotation of a nut 156 pulls the ADR endplate from the spine. The puller tool provides counter traction.

FIGS. 1E-1H show the way in which single tools or multiple tools may be inserted between implanted endplates for ADR removal according to this invention. Shape-memory materials or materials that curve after insertion may be useful for such purpose. FIG. 1H shows the way in which the end of a tool may cooperate with a depression 180 in the vertebral endplate 182 for removal purposes.

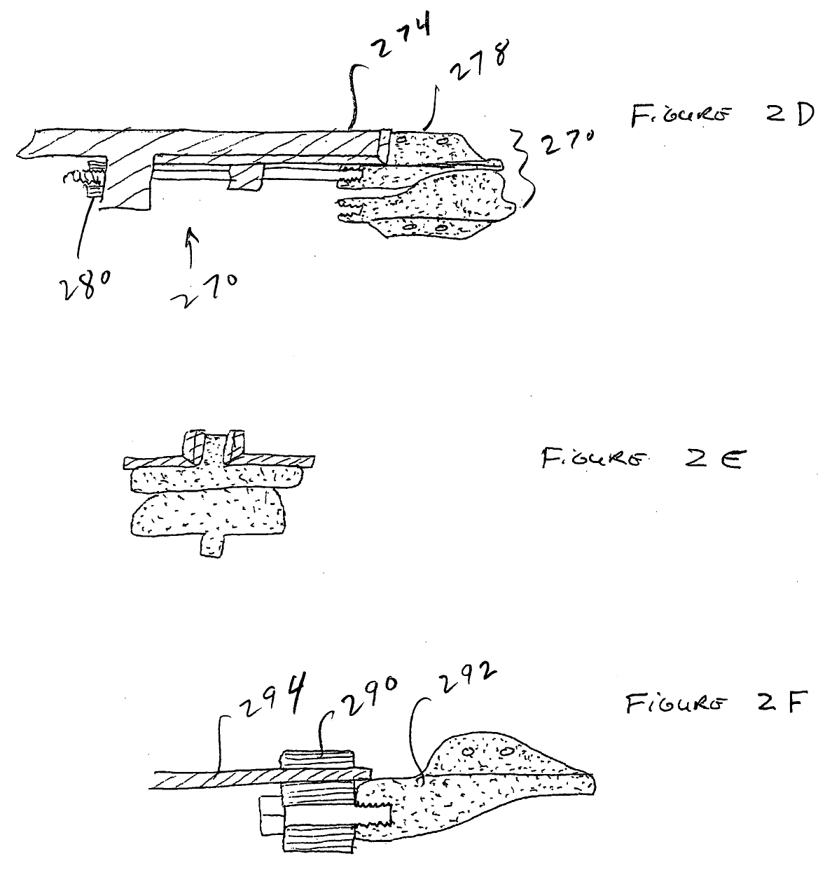

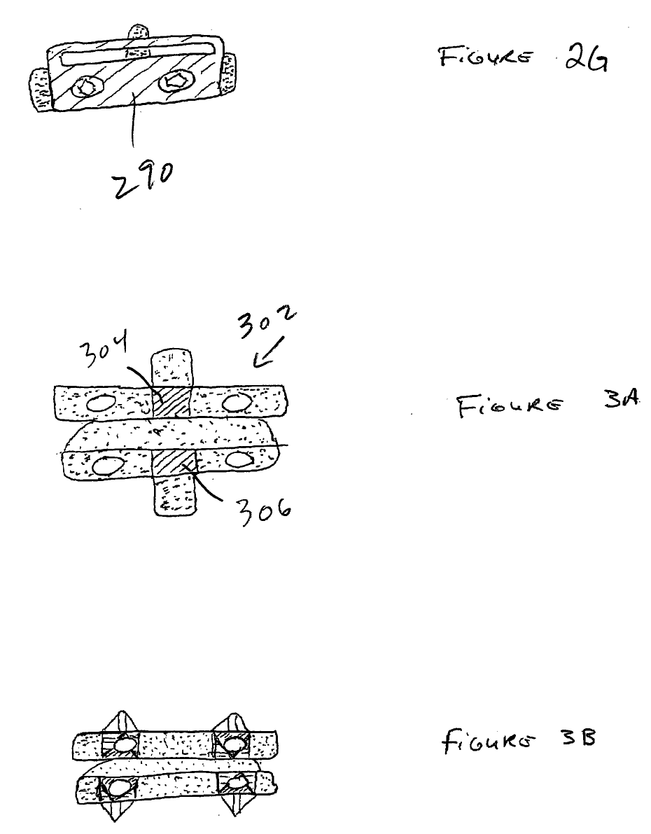

FIG. 2A is a sagittal cross section of an ADR and a guided chisel. The chisel 202 is forced over the shaft 204 of a tool threaded into the ADR, by turning a nut 206. The chisel (or osteotome) 202 is guided between the vertebra and the surface of the ADR.

FIG. 2B is the view of the top of an ADR 220 with a keel 222 and a novel instrument 224 designed to fit around the keel. The chisel or osteotome has mechanisms to prevent the instrument from entering the spinal canal. First, the slot 226 within the tool is designed to impinge against the keel of the ADR before the blades of the tool enter the spinal canal. The tool has been introduced with an anterior approach and portions 230, 232 of the ADR are obscured. Second, the tool has projections 240, 242 from the side of the tool that impinge against the vertebrae or the disc (not shown), before the cutting edge of the tool enters the spinal canal. FIG. 2C is a view of the top of an ADR with two rows of spikes 260 and an embodiment of the tool drawn in FIG. 2B designed to cut around the spikes.

FIG. 2D is a sagittal cross section of an ADR 270, a guide 272, and a tool 274 designed to cut around the top of the ADR and the sides of a keel 278. The cutting edge of the tool 274 is L shaped to fit the top of the ADR. Similar to device drawn in FIG. 2A, rotating the nut 280 on the device forces the cutting tool between the ADR and the vertebrae. FIG. 2E is a coronal cross section of an ADR and the cutting tool draw in FIG. 2D.

FIG. 2F is a sagittal cross section of an ADR and an alternative cutting tool and cutting guide. The cutting guide 290 is attached to the ADR 292 using bolts 294. Other methods of attaching the cutting guide are acceptable. A saw blade, burr, osteotome, chisel, or other cutting instrument 294 is directed between the ADR and the vertebrae. FIG. 2G is view of the front of the superior endplate of an ADR and the embodiment of the cutting guide drawn in FIG. 2F.

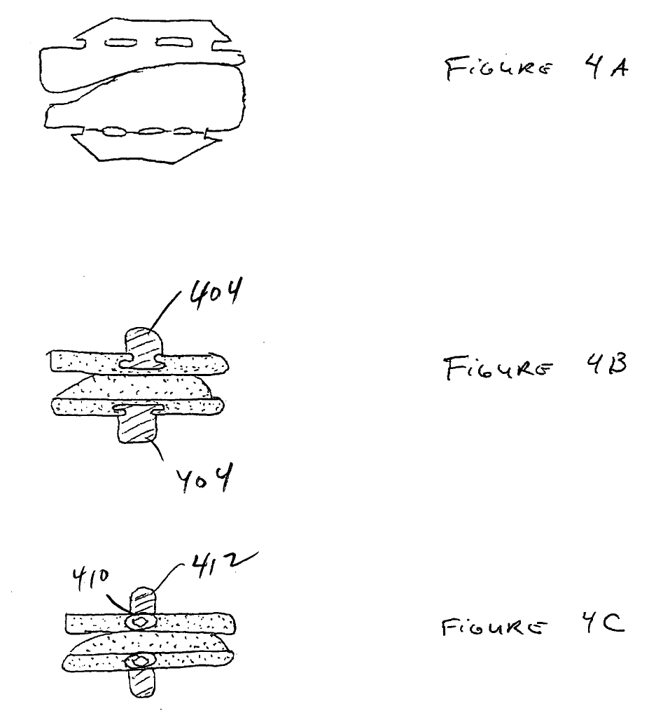

FIG. 3A is the view of the front of an ADR 302 with novel marks that locate the size, type, and position of projections from the ADR. The markings on the ADR are represented by areas 304, 306. The markings indicate the width and location of keels in this embodiment of the invention. The white circles in the ADR represent holes to attach extraction tools or cutting guides. FIG. 3B is the view of the front of an ADR with markings to indicate the size and location of ADR spikes.

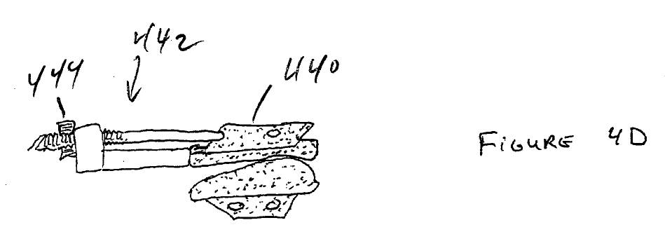

FIG. 4A is a lateral view of an ADR with novel stress risers to facilitate separation of projections from the ADR. In this embodiment of the invention, holes, notches, and grooves are used to create the stress risers where keels attach to the ADR. Similar stress risers can be used for other types of ADR projections. FIG. 4B is a coronal cross section of an ADR with novel modular keels. The keels 404 fit in grooves in the ADR endplates. FIG. 4C is a view of the front of the ADR drawn in FIG. 4B. Screws 410 are used to hold the modular keels 412 in the ADR endplates. The screws can have threads that deform slightly to lock the screws in the ADR endplates. FIG. 4D is a view of the side of an ADR with modular keels 440 and a keel extracting instrument 442. A nut 444 is rotated around the shaft of an instrument that is attached to the front of the keel. The keel is extracted from the front of the ADR as the nut is tightened.

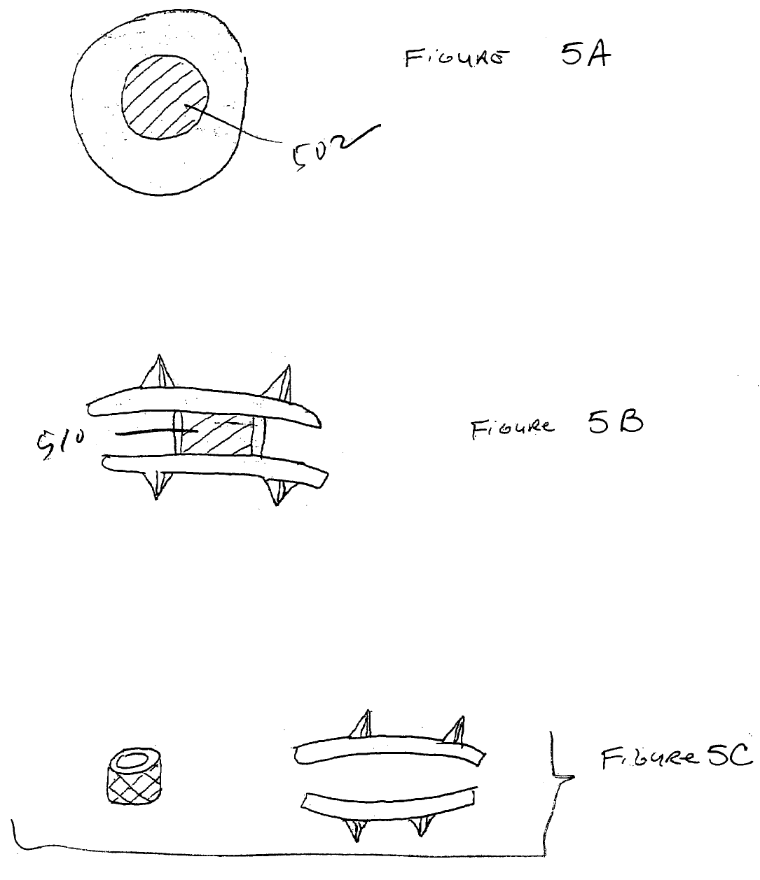

FIG. 5A is a view of the top of the bottom ADR endplate or the view of the bottom of the top endplate including an area 502 which represents a removable portion of the ADR endplate. The section of the endplate could be removed to expose the underlying bone of the vertebrae. Exposing the underlying bone would be helpful if the cushioning component of the ADR was removed to perform a spinal fusion. FIG. 5B is a view of the side of the embodiment of the ADR drawn in FIG. 5A. A cage 510 filled with bone or bone growth promoting material is positioned into or around the opening in the ADR endplates. FIG. 5C is an exploded view of the embodiment of the ADR drawn in FIGS. 5A and 5B. The small circles to the left of the drawing represent bone graft.

I claim:

1. An intervertebral implant system, comprising:
an artificial disc replacement (ADR) adapted for attachment to a vertebral body; and
apparatus specifically directed to detaching the ADR from the vertebral body.

2. The system of claim 1, wherein the apparatus includes:
an ADR providing the first portion of a connector; and
an instrument providing the second portion of the connector, enabling the instrument to temporarily attached to the ADR for removal purposes.

3. The system of claim 2, wherein the instrument further includes one or more portions that bear against a vertebral body during the detachment process.

4. The system of claim 1, wherein the apparatus includes a guide to direct a cutting implement between the ADR and a vertebral body.

5. The system of claim 1, wherein:
the ADR includes a keel; and
the apparatus includes comprising a tool designed to cut around the keel.

6. The system of claim 1, wherein:
the ADR includes one or more projections; and
marks to indicate the size, type or position of the projections.

7. The system of claim 1, wherein the ADR includes stress risers to facilitate the separation of the ADR from a vertebral body.

8. The system of claim 1, wherein the apparatus includes a curved instrument that reaches to an opposing side of the ADR for removal purposes.
